(19) United States
(12) Patent Application Publication
(10) Pub. No.: US 2010/0203118 A1
(43) Pub. Date: Aug. 12, 2010

(54) COLLAPSIBLE WATER-CONTAINING CAPSULES

(76) Inventors: Kojo Tanaka, Ashiya (JP); Takashi Sako, Kobe (JP)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/701,084

(22) Filed: Feb. 5, 2010

Related U.S. Application Data
(60) Provisional application No. 61/150,427, filed on Feb. 6, 2009.

(51) Int. Cl. A61K 8/02; A61K 8/29; A61Q 1/00

(52) U.S. Cl. 424/451; 424/62; 424/63

(57) ABSTRACT

Collapsible water-containing capsules comprising by weight: (a) from about 40% to about 95% of a water phase the water phase comprising at least 50% water by weight of the water phase; and (b) from about 5% to about 20% of a spindle-shaped metal oxide powder which is hydrophobically surface-treated and has an average long axis particle size of from about 25 nm to about 150 nm, an average short axis particle size of from about 4 nm to about 50 nm, and an aspect ratio of greater than about 3.

### CROSS REFERENCE TO RELATED APPLICATIONS

This application claims the benefit of U.S. Provisional Application No. 61/150,427 filed on Feb. 6, 2009.

### FIELD OF THE INVENTION

The present invention relates to a collapsible water-containing capsule which is stable under normal storage conditions as well as normal mixing processes, however, collapses upon application on the personal surface. The present invention further relates to methods of making such capsules, personal care compositions utilizing such capsules, and method of treating or make-up of the skin using such capsules.

### BACKGROUND

A foundation composition can be applied to the face and other parts of the body to even skin tone and texture and to hide pores, imperfections, fine lines and the like. A foundation composition is also applied to moisturize the skin, to balance the oil level of the skin, and to provide protection against the adverse effects of sunlight, wind, and other environmental factors.

Foundation compositions are generally available in the form of liquid or cream suspensions, emulsions, gels, pressed powders, loose powders or anhydrous oil and wax compositions. Emulsion-type foundations are suitable in that they provide moisturizing effects by the water and water-soluble skin treatment agents incorporated. On the other hand, a larger amount and variation of powders and pigments can be formulated into pressed powders and loose powders.

Recently, consumers who seek moisturization as well as the ideal look having both good coverage and natural look on the skin, have the habit of a two step regimen of foundation application. The two step regimen typically contains application of a liquid or emulsion form foundation followed by a pressed or loose powder foundation. It is conceived by such demanding consumers that such two-step regimen provides best results, however, such regimen is also quite elaborate. There is a need for a foundation product which can provide both good feel and good appearance on the skin.

Meanwhile, collapsible water-containing capsules are known in the art. Such capsules provide a unique feel or change of feel upon application and collapsing on the skin. Upon application to the skin, such capsules provide a moisturizing or fresh feeling. Such capsules may also deliver water-soluble skin active agents such as vitamin C derivatives to the skin, in a more or less stable manner.

Known collapsible water-containing capsules are typically made of fine porous powders such as silica particles which may or may not be surface treated, as disclosed in, for example, PCT Publication WO 01/85138, Japanese Patent Publications 2001-131528A, 2000-247823A, 2000-309506A, 11-130614A, 10-265367A, 5-65212A, and 4-308520A. While the use of porous silica may provide a relatively stable capsule, it has also been observed that porous silica may give a negative dry feeling after application on the skin. This is obviously not preferred for a product that is expected to provide a moisturizing feel due to abundant water contained in the capsule. Further, some of these publications disclose extreme conditions and steps for making the capsules, including high shear mixing and freezing prior to shearing. Such conditions and steps are costly and unfavorable from a commercial point of view.

Some attempts have been made to utilize powders coated by fluorine surface coating agents such as disclosed in Japanese Patent Publications 2006-509732A, 2001-226230A, 2001-158716A, and 1-125314A. None of the above mentioned capsules, however, provide a favorable application to the skin while also providing satisfactory shear stress tolerance. PCT Publication WO 2008/018028 discloses capsules made of powders coated by fluorine surface coating agents. It would be of particular advantage, from a safety and environmental point of view, to provide capsules that are devoid of fluorine surface coating agents.

Based on the foregoing, there is a need for a collapsible water-containing capsule which is capable of providing good feel to the personal surface, while having appropriate shear tolerance such that it is stable under normal storage conditions as well as normal mixing processes, however, collapses upon a certain shear stress upon application on the personal surface. There is further a need for a collapsible water-containing capsule which provides good appearance on the personal surface. There is further a need for a collapsible water-containing capsule which can be manufactured economically.

None of the existing art provides all of the advantages and benefits of the present invention.

### SUMMARY

The present invention is directed to a collapsible water-containing capsule comprising by weight:
(a) from about 40% to about 95% of a water phase comprising at least 50% water by weight of the water phase; and
(b) from about 5% to about 20% of a spindle-shaped metal oxide powder which is hydrophobically surface-treated and has an average long axis particle size of from about 25 nm to about 150 nm, an average short axis particle size of from about 4 nm to about 50 nm, and an aspect ratio of greater than about 3.

The present invention is also directed to personal care compositions comprising the aforementioned collapsible water-containing capsule.

The present invention is also directed to a process for making the aforementioned collapsible water-containing capsule.

These and other features, aspects, and advantages of the present invention will become evident to those skilled in the art from a reading of the present disclosure with the appended claims.

### BRIEF DESCRIPTION OF THE DRAWINGS

While the specification concludes with claims particularly pointing out and distinctly claiming the invention, it is believed that the present invention will be better understood from the following description of preferred, nonlimiting embodiments and representations taken in conjunction with the accompanying drawings in which:

FIG. 1 is a microscopic photograph of a preferred embodiment of the present collapsible water-containing capsule, along with a scale showing the length of 100 μm.

### DETAILED DESCRIPTION

While the specification concludes with claims particularly pointing out and distinctly claiming the invention, it is believed that the present invention will be better understood from the following description.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

### Collapsible Water-Containing Capsule

The present invention is related to a collapsible water-containing capsule which comprises, by weight of the capsule, from about 40% to about 95% of a water phase, among which all can be water, and may further contain water-soluble solvents and gelling agents. To hold such abundant amount of water in the structure, the capsule of the present invention comprises a spindle-shaped metal oxide powder which is hydrophobically surface-treated and has an average long axis particle size of from about 25 nm to about 150 nm, an average short axis particle size of from about 4 nm to about 50 nm, and an aspect ratio of greater than about 3. The present invention provides a collapsible water-containing capsule which is stable under normal storage conditions as well as normal mixing processes, however, collapses upon application.

Without being bound by theory, it is believed that the hydrophobically surface-treated spindle-shaped metal oxide powder provides a fractal structure surrounding and repelling the water phase, while also maintaining balanced adhesion with each other, and thereby provides the stability and integrity of the capsule.

Preferably, the present capsule is substantially free of surfactant. Without being bound by theory, it is believed that surfactants negatively affect the stability and shear stress tolerance of the present capsule by decreasing the surface tension difference between the water phase and the hydrophobically surface-treated spindle-shaped metal oxide. Herein, surfactants include those which have detersive capability, as well as those which only act as emulsifiers for emulsifying water and oil phases.

Preferably, the present capsule comprises less than 1% of porous powders having a particle size of less than 1 μm, more preferably substantially free of porous powders having a particle size of less than 1 μm. Without being bound by theory, it is believed that porous powders of small size absorb sebum from the personal surface to such an extent that a dry negative feeling is provided to the personal surface. Porous powders preferably not comprised at 1% or more in the present invention include silica, aluminum oxide, calcium carbonate, cellulose, and others that may have a porous structure when observed under magnification. It is noted that powders made from the same chemical compound may take either a porous or non-porous structure, based on the process it is purified, processed, synthesized, or otherwise treated.

Preferably, the present capsule is substantially free of fluorine surface coated pigments, for addressing safety and environmental concerns. Materials for fluorine surface coating that are preferably avoided herein include perfluorooctyl triethoxysilane, perfluoroalkyl phosphoric acid, their salts, and mixtures thereof.

The collapsible water-containing capsule of the present invention provides unique benefits on the personal surface, such as skin, hair, or scalp, when collapsed on the surface. It provides an initially fresh, and then moisturizing feel to the surface, by releasing the abundant water in the capsule. The capsule further provides a good feel to the surface by the characteristic of the hydrophobically surface-treated spindle-shaped metal oxide. Additional feel benefits can be provided by containing other powders to the capsule. When the powder components are applied on the surface, the components provide the appearance benefits inherent of such powder components.

The present capsule may, by itself, provide a product in the form of a loose powder product. The present capsule may also be mixed with other components to provide different product forms. The present capsule has appropriate shear tolerance such that it is stable under normal storage conditions, as well as normal mixing process, for example when mixing with the other components, however, collapses upon application to the personal surface.

The present capsule is particularly useful as personal care compositions for delivering water, the powders, and other components to personal surface. Personal care compositions herein include those for the purpose of skin care, make-up, extensive treatment, perfume, antiperspiration, deodorizing, hair coloring, hair treatment, hair styling, and others. Personal care compositions herein can take the product form of powders, wax solidified solid forms, liquids, lotions, pastes, aerosols, and others. One highly preferred product form embodiment is powder for use on the skin, such as foundation and skin care products.

The present capsule is particularly suitable for using as or incorporating in personal care compositions for treatment of the skin, and make-up of the skin. Accordingly, the present invention is also related to a method of treating or making up of the skin comprising the steps of:
(1) providing the collapsible water-containing capsule of the present invention;
(2) shearing the collapsible water-containing capsule on the skin by a finger or an applicator to allow the collapsible water-containing capsule to collapse; whereby the components of the collapsible water-containing capsule are applied on the skin; and
(3) allowing the water to evaporate and/or be absorbed in the skin.

For such personal skin care compositions, the powder components of the present capsule are selected to provide the appropriate skin treatment and/or make-up benefits. Further, the present capsule may comprise various skin benefit agents and perfumes in a dissolved or dispersed form in the water phase or attracted within the powder components. It is advantageous to deliver such skin benefit agents, and perfumes encompassed in the present collapsible water-containing capsule, for one or more reasons. For those components that are heat sensitive, the present capsule prevents or delays evaporation prior to use. For those components that may be deteriorated or compromised in benefit by coming to contact with the remainder of the personal care composition, the present capsule acts as a barrier. Other components may provide a certain sensation upon application and collapsing of the present capsule.

### Water Phase

The present capsule comprises a water phase, the water phase comprising at least 50%, preferably at least 60%, water by weight of the water phase, optional water-soluble solvent, and optional gelling agent, detailed hereafter. The present capsule comprises, by weight of the capsule, from about 40% to about 95%, preferably from about 60% to about 90%, of the water phase. The water phase may be made only by water. Deionized water is preferably used. Water from natural sources including mineral cations may also be used, depending on the desired characteristic of the product. In one preferred embodiment, water may be sourced from fermented biological cultures or its filtrates. A highly preferred commercial source of this kind is Galactomyces ferment filtrate by the tradename SK-II Pitera available from Kashiwayama.

The pH of the water phase is selected in view of the desired characteristic of the product, and particularly, when skin benefit agents are included, the activity and stability of the skin benefit agents. In one preferred embodiment the pH is adjusted to from about 4 to about 8. Buffers and other pH adjusting agents can be included to achieve the desirable pH.

### Water-Soluble Solvent

The water phase of the present capsule may further comprise a water-soluble solvent selected from lower alkyl alcohols and water-soluble humectants. The water-soluble solvents are selected according to the desired skin feel to be delivered, and/or for delivering certain skin benefit agents.

Lower alkyl alcohols useful herein are monohydric alcohols having 1 to 6 carbons, more preferably ethanol and isopropanol.

Water soluble humectants useful herein include polyhydric alcohols such as butylene glycol (1,3 butanediol), pentylene glycol (1,2-pentanediol), glycerin, sorbitol, propylene glycol, hexylene glycol, ethoxylated glucose, 1,2-hexane diol, hexanetriol, dipropylene glycol, erythritol, trehalose, diglycerin, xylitol, maltitol, maltose, glucose, fructose; and other water-soluble compounds such as urea, sodium chondroitin sulfate, sodium hyaluronate, sodium adenosin phosphate, sodium lactate, pyrrolidone carbonate, cyclodextrin, and mixtures thereof. Also useful herein include water soluble alkoxylated nonionic polymers such as polyethylene glycols and polypropylene glycols having a molecular weight of up to about 1000 such as those with CTFA names PEG-200, PEG-400, PEG-600, PEG-1000, and mixtures thereof.

In one preferred embodiment, the present capsule comprises from about 1% to about 30% of a water-soluble humectant. In one highly preferred embodiment wherein the capsule is used as a foundation, the capsule comprises from about 3% to about 30% of a water-soluble humectant.

Commercially available humectants herein include: butylene glycol with tradename 1,3-Butylene glycol available from Celanese, pentylene glycol with tradename HYDROLITE-5 available from Dragoco, glycerin with tradenames STAR and SUPEROL available from The Procter & Gamble Company, CRODEROL GA7000 available from Croda Universal Ltd., PRECERIN series available from Unichema, and a same tradename as the chemical name available from NOF; propylene glycol with tradename LEXOL PG-865/855 available from Inolex, 1,2-PROPYLENE GLYCOL USP available from BASF; sorbitol with tradenames LIPONIC series available from Lipo, SORBO, ALEX, A-625, and A-641 available from ICI, and UNISWEET 70, UNISWEET CONC available from UPI; dipropylene glycol with the same tradename available from BASF; diglycerin with tradename DIGLYCEROL available from Solvay GmbH; xylitol with the same tradename available from Kyowa and Eizai; maltitol with tradename MALBIT available from Hayashibara, sodium chondroitin sulfate with the same tradename available from Freeman and Bioiberica, and with tradename ATOMERGIC SODIUM CHONDROITIN SULFATE available from Atomergic Chemetals; sodium hyaluronate available from Chisso Corp, the same with tradenames ACTIMOIST available from Active Organics, AVIAN SODIUM HYALURONATE series available from Intergen, HYALURONIC ACID Na available from Ichimaru Pharcos; sodium adenosin phophate with the same tradename available from Asahikasei, Kyowa, and Daiichi Seiyaku; sodium lactate with the same tradename available from Merck, Wako, and Showa Kako, cyclodextrin with tradenames CAVITRON available from American Maize, RHODOCAP series available from Rhone-Poulenc, and DEXPEARL available from Tomen; and polyethylene glycols with the tradename CARBOWAX series available from Union Carbide.

### Gelling Agents

The water phase of the capsule of the present composition may further comprise, by weight of the capsule, from about 0.1% to about 20%, preferably from about 0.1% to about 5%, of a gelling agent that provides the water phase a viscosity of from about 10 mPas to about 1,000,000 mPas, preferably from about 10 mPas to about 100,000 mPas. The gelling agent holds water and optional water-soluble solvents in a relatively rigid structure, and thereby believed to improve the stability and integrity of the capsule, such that the shelf life of the capsule is prolonged.

The polymers useful as the gelling agent herein are water soluble or water miscible polymers. The term “water soluble or water miscible” with regard to the gelling agents herein, relate to compounds that are dissolved to make a transparent solution when dissolved in ample amount of water with or without the aid of elevated temperature and/or mixing.

Useful herein are starch derivative polymers such as carboxymethyl starch, and methylhydroxypropyl starch. Commercially available compounds that are highly useful herein include sodium carboxymethyl starch with tradename COVAGEL available from LCW.

Useful herein are cellulose derivative polymers. Cellulose derivative polymers useful herein include methylcellulose, ethylcellulose, hydroxyethylcellulose, hydroxyethyl ethylcellulose, hydroxypropyl methyl cellulose, nitrocellulose, sodium cellulose sulfate, sodium carboxymethylcellulose, crystalline cellulose, cellulose powder, and mixtures thereof. Also useful are starch derivative polymers such as carboxymethyl starch, and methylhydroxypropyl starch. Commercially available compounds that are highly useful herein include hydroxyethylcellulose with tradename Natrosol Hydroxyethylcellulose, and carboxymethylcellulose with tradename Aqualon Cellulose Gum, both available from Aqualon.

Useful herein are carboxylic acid/carboxylate copolymers. Commercially available carboxylic acid/carboxylate copolymers useful herein include: CTFA name Acrylates/C10-30 Alkyl Acrylate Crosspolymer having tradenames Pemulen TR-1, Pemulen TR-2, Carbopol 1342, Carbopol 1382, and Carbopol ETD 2020, all available from B. F. Goodrich Company.

Neutralizing agents may be included to neutralize the carboxylic acid/carboxylate copolymers herein. Nonlimiting examples of such neutralizing agents include sodium hydroxide, potassium hydroxide, ammonium hydroxide, monoethanolamine, diethanolamine, triethanolamine, diisopropanolamine, aminomethylpropanol, tromethamine, tetrahydroxypropyl ethylenediamine, and mixtures thereof.

Polyalkylene glycols having a molecular weight of more than about 1000 are useful herein. Useful are those having the following general formula:

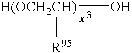

wherein R⁹⁵ is selected from the group consisting of H, methyl, and mixtures thereof. When R⁹⁵ is H, these materials are polymers of ethylene oxide, which are also known as polyethylene oxides, polyoxyethylenes, and polyethylene glycols. When R⁹⁵ is methyl, these materials are polymers of propylene oxide, which are also known as polypropylene oxides, polyoxypropylenes, and polypropylene glycols. When R⁹⁵ is methyl, it is also understood that various positional isomers of the resulting polymers can exist. In the above structure, x3 has an average value of from about 1500 to about 25,000, preferably from about 2500 to about 20,000, and more preferably from about 3500 to about 15,000. Other useful polymers include the polypropylene glycols and mixed polyethylene-polypropylene glycols, or polyoxyethylene-polyoxypropylene copolymer polymers. Polyethylene glycol polymers useful herein are PEG-2M wherein R⁹⁵ equals H and x3 has an average value of about 2,000 (PEG-2M is also known as Polyox WSR® N-10, which is available from Union Carbide and as PEG-2,000); PEG-5M wherein R⁹⁵ equals H and x3 has an average value of about 5,000 (PEG-5M is also known as Polyox WSR® N-35 and Polyox WSR® N-80, both available from Union Carbide and as PEG-5,000 and Polyethylene Glycol 300,000); PEG-7M wherein R⁹⁵ equals H and x3 has an average value of about 7,000 (PEG-7M is also known as Polyox WSR® N-750 available from Union Carbide); PEG-9M wherein R⁹⁵ equals H and x3 has an average value of about 9,000 (PEG 9-M is also known as Polyox WSR® N-3333 available from Union Carbide); and PEG-14 M wherein R⁹⁵ equals H and x3 has an average value of about 14,000 (PEG-14M is also known as POLYOX WSR® N-3000 available from Union Carbide).

Useful herein are vinyl polymers such as cross linked acrylic acid polymers with the CTFA name Carbomer, pullulan, mannan, scleroglucans, polyvinylpyrrolidone, polyvinyl alcohol, guar gum, hydroxypropyl guar gum, xanthan gum, acacia gum, arabia gum, tragacanth, galactan, carob gum, karaya gum, locust bean gum, carrageenin, pectin, amylopectin, agar, quince seed (Cyclonia oblonga Mill), starch (rice, corn, potato, wheat), algae colloids (algae extract), microbiological polymers such as dextran, succinoglucan, starch-based polymers such as carboxymethyl starch, methylhydroxypropyl starch, alginic acid-based polymers such as sodium alginate, alginic acid propylene glycol esters, acrylate polymers such as sodium polyacrylate, polyacrylamide, polyethyleneimine, and inorganic water soluble material such as bentonite, aluminum magnesium silicate, laponite, hectonite, and anhydrous silicic acid.

Commercially available gelling agents useful herein include xanthan gum with tradename KELTROL series available from Kelco, Carbomers with tradenames CARBOPOL 934, CARBOPOL 940, CARBOPOL 950, CARBOPOL 980, and CARBOPOL 981, all available from B. F. Goodrich Company, acrylates/steareth-20 methacrylate copolymer with tradename ACRYSOL 22 available from Rohm and Hass, polyacrylamide with tradename SEPIGEL 305 available from Seppic, sodium polyacrylate with tradename COVACRYL MV60 available from LCW, glyceryl polymethacrylate with tradename LUBRAGEL NP, and a mixture of glyceryl polymethacrylate, propylene glycol and PVM/MA copolymer with tradename LUBRAGEL OIL available from ISP, scleroglucan with tradename Clearogel SC11 available from Michel Mercier Products Inc. (NJ, USA), ethylene oxide and/or propylene oxide based polymers with tradenames CARBOWAX PEGs, POLYOX WASRs, and UCON FLUIDS, all supplied by Amerchol.

Useful herein are amphoteric polymers such as Polyquaternium 22 with tradenames MERQUAT 280, MERQUAT 295, Polyquaternium 39 with tradenames MERQUAT PLUS 3330, MERQUAT PLUS 3331, and Polyquaternium 47 with tradenames MERQUAT 2001, MERQUAT 2001N, all available from Calgon Corporation. Other useful amphoteric polymers include octylacrylamine/acrylates/butylaminoethyl methacrylate copolymers with the tradenames AMPHOMER, AMPHOMER SH701, AMPHOMER 28-4910, AMPHOMER LV71, and AMPHOMER LV47 supplied by National Starch & Chemical.

### Spindle-Shaped Metal Oxide Powder

The collapsible water-containing capsule of the present composition comprises, by weight of the capsule, from about 5% to about 20%, preferably from about 7% to about 18%, of a spindle-shaped metal oxide powder which is hydrophobically surface-treated and has an average long axis particle size of from about 25 nm to about 150 nm, preferably from about 30 nm to about 100 nm, an average short axis particle size of from about 4 nm to about 50 nm, preferably from about 5 nm to about 20 nm, and an aspect ratio of greater than about 3, preferably from about 4 to about 50. With regard to the spindle-shaped metal oxide powder, what is meant by “average particle size” is the arithmetic average by observing the particles with an transmission electron microscope, and what is meant by “aspect ratio” is the ratio of the long axis to the short axis. The spindle-shaped metal oxide powder of the present invention is distinguished from other metal oxide powders useful in the art, the other metal oxide powders being more or less amorphous in shape, and thus having an aspect ratio of less than 3. The metal oxide is preferably selected from titanium dioxide, zinc oxide and iron oxide, more preferably titanium dioxide. The coating materials useful for hydrophobic surface-treating of the spindle-shaped metal oxide powder include dimethyl polysiloxane, methyl hydrogen polysiloxane, methyl phenyl polysiloxane, n-octyl triethoxy silane, methyl-alpha-styrene polysiloxane, acryl silicone copolymer, and mixtures thereof.

Without being bound by theory, it is believed that, by the surface tension of the hydrophobic surface of the spindle-shaped metal oxide powder, the spindle-shaped metal oxide powders align at the phase boundary of the water phase binding with each other via van-der-Waals binding, while the high aspect ratio shape provides a fractal structure surrounding and repelling the water phase. It is further believed that the overall structure due to the hydrophobic surface, combined with the relatively small particle size of the spindle-shaped metal oxide powder, contributes to the suitable shear stress tolerance of the collapsible water-containing capsule of the present composition. In addition to constructing the core structure of the collapsible water-containing capsule, the spindle-shaped metal oxide powders may also provide benefits such as coverage to the skin, and UV protection to the skin.

Commercially available spindle-shaped metal oxide powders herein include Titanium Dioxide coated with triethoxycaprylylsilane having a long axis particle size of about 60 nm and a short axis particle size of about 10 nm (aspect ratio about 6) with tradename OTS-11 TTO-V-3 available from Daito Kasei.

### Spherical Powder

The collapsible water-containing capsule of the present composition may further comprise, by weight of the capsule, from about 0.1% to about 40%, preferably from about 3% to about 25%, of a spherical powder. The spherical powder herein has a particle size of at least 1 μm, preferably from about 1 μm to about 25 μm, more preferably from about 4 μm to about 15 μm, has a hydrophobic surface, and is spherical in shape. The spherical powders may be coated with the same coating material described above for the spindle-shaped metal oxide powder.

Without being bound by theory, it is believed that, due to the larger size and spherical shape of the spherical powder, the spherical powder aligns at the outer boundary of the spindle-shaped metal oxide powder. It is believed that the dual covered structure thus provided by the spindle-shaped metal oxide powder and spherical powders provide improved shear stress tolerance to the present capsule.

The spherical powder also provides a unique appearance effect or skin feel that is not easily delivered by only the spindle-shaped metal oxide powder. In one example, the spindle-shaped metal oxide powders alone may provide a relatively matte finish and emphasize, rather than hide, skin unevenness such as pores. A spherical and translucent spherical powder can improve the natural appearance by light diffusion effect due to its shape and translucency. In another example, the spindle-shaped metal oxide powders alone may provide a squeaky feel on the skin due to their small size. A soft spherical spherical powder may alleviate such skin feel and provide good smooth feel.

The spherical powders useful herein include; polyacrylates, silicates, sulfates, alumina, metal dioxides, carbonates, celluloses, polyalkylenes, vinyl acetates, polystyrenes, polyamides, acrylic acid ethers, silicones, and mixtures and complexes thereof. Specifically, materials useful herein include polyacrylates such as methyl methacrylate copolymer and nylon, cross linked polymethyl methacrylate; silicates such as calcium silicate, magnesium silicate, barium silicate, aluminium silicate and silica beads; alumina; metal dioxides such as titanium dioxide and aluminium hydroxide; carbonates such as calcium carbonate, magnesium carbonate; celluloses; polyalkylenes such as polyethylene, and polypropylene; vinyl acetates; polystyrenes; polyamides; acrylic acid ethers such as acrylic acid methyl ether and acrylic acid ethyl ether; polyvinyl pyrrolidones; and silicones such as polyorganosilsesquioxane resin such as polymethyl silsequioxane and solid silicone elastomers such as vinyl dimethicone/methicone silsesquioxane crosspolymer. Highly preferred materials are selected from the group consisting of methyl methacrylate copolymer, silica beads, nylon, polymethyl silsesquioxane, vinyl dimethicone/methicone silsesquioxane crosspolymer, polyorganosiloxane elastomer, and mixtures thereof.

In one embodiment, polyorganosilsesquioxane resin and silicone elastomer powders may be used for enhancing the effect of hiding skin pores.

Commercially available spherical powders highly useful herein include methyl methacylate copolymer with tradename GANZ PEARL series available from Ganz Chemical Co., Ltd., and SYLYSIA series available from Fuji Sylysia Chemical, Nylon-12 with tradename NYLON POWDER series available from Toray Dow Corning, Nylon-12 coated with C₉₋₁₅ fluoroalcohol phosphates (5 μm) with tradename PF-5 NYLON SP 500 available from Daito Kasei, polymethyl silsesquioxiane coated with C₉₋₁₅ fluoroalcohol phosphates with tradename PF-5 TOSPEARL 145 available from Daito Kasei, vinyl dimethicone/methicone silsesquioxane crosspolymer with tradenames KSP series available from ShinEtsu Chemical Co., Ltd., Tokyo Japan, and hardened polyorgano siloxane elastomers with tradenames TREFIL series available from Toray Dow Corning.

In one highly preferred embodiment, the spherical powder is a water repelling silicone elastomer powder comprising 100 weight parts of a spherical silicone elastomer particle and 0.5-25 weight parts of polyorganosilsequioxane for coating the spherical silicone elastomer particle; wherein the water repelling silicone elastomer powder does not disperse in, but floats in water; has an average particle size of at least 1 μm and has a softness of from about 10 to about 80 measured by Durometer A Hardness, preferably the surface of the coated polyorganosilsequioxane is further bonded with a trimethylsilyl group, and preferably surface of the coated polyorganosilsequioxane is further condensated by hydrolyzing with tetraalkoxysilane and at least one silylation agent selected from the group consisting of, trimethylalkoxysilane, trimethylsilanol, and hexamethyldisilazine. Such water repelling silicone elastomer powder is particularly advantageous for providing stability to the capsule. Such water repelling silicone elastomer powder is exemplified as Reference Example 1 below.

### Color Powder

The collapsible water-containing capsule of the present composition may further comprise, by weight of the capsule, from about 0.1% to about 8%, preferably from about 0.5% to about 5%, of a color powder. For one highly preferred embodiment of the present invention, the present capsule is a foundation comprising color powder. The color powder herein has a particle size of from about 0.15 μm to less than 1 μm, preferably from about 150 nm to about 500 nm, and is surface coated with a hydrophobic coating material. The color powder may be coated with the same coating material described above for the spindle-shaped metal oxide powder.

The color powders useful herein include those that provide color or change tone, and also those that provide a certain skin feel. The color powders useful herein include alumina, barium sulfate, calcium secondary phosphate, zirconium oxide, zinc oxide, hydroxy apatite, titanium dioxide, iron oxide, iron titate, ultramarine blue, Prussian blue, chromium oxide, chromium hydroxide, cobalt oxide, cobalt titanate, titanium dioxide coated mica boron nitride; organic powders such as polyester, polyethylene, polystyrene, methyl methacrylate resin, 12-nylon, 6-nylon, styrene-acrylic acid copolymers, poly propylene, vinyl chloride polymer, tetrafluoroethylene polymer, fish scale guanine, laked tar color dyes, and laked natural color dyes. Particularly useful herein as the color powder are titanium dioxide, zinc oxide, iron oxide, barium sulfate, and mixtures thereof.

Commercially available color powders highly useful herein include Titanium Dioxide coated with triethoxycaprylsilane having a particle size of about 250 nm with tradename OTS-2 TIO2 CR-50 available from Daito Kasei, yellow, black and red iron oxide coated with Triethoxycaprylylsilane having a particle size of about 400 nm with tradenames OTS-2 YELLOW LL-100P, OTS-2 BLACK BL-100P, and OTS-2 RED R-516P available from Daito Kasei.

### Skin Benefit Agent

The capsule of the present composition may further comprise a skin benefit agent dissolved or dispersed in the water phase or the powder components. Those skin benefit agents of polar nature can be dissolved or dispersed in the water phase, while those that do not dissolve or disperse in the water phase may be mixed and attracted within the powder components. When included, the skin benefit agent is included in an amount that does not affect the stability of the capsule, typically by weight of the capsule, at from about 0.001% to about 20%.

The skin benefit agents useful herein include skin lightening agents, anti-acne agents, emollients, non-steroidal anti-inflammatory agents, topical anaesthetics, artificial tanning agents, antiseptics, anti-microbial and anti-fungal actives, skin soothing agents, UV protection agents, skin barrier repair agents, anti-wrinkle agents, anti-skin atrophy actives, lipids, sebum inhibitors, skin sensates, protease inhibitors, skin tightening agents, anti-itch agents, hair growth inhibitors, desquamation enzyme enhancers, anti-glycation agents, antiperspirant actives, oxidative hair colorants, hair styling agents, and mixtures thereof.

Commercially available flavonoid compounds include hesperidin, methylhesperidin, and rutin available from Alps Pharmaceutical Industry Co. Ltd. (Japan); and glucosyl hesperidin with tradename alpha-Ghesperidin PS-CC and glucosyl rutin available from Hayashibara Biochemical Laboratories, Inc. (Japan) and Toyo Sugar Refining Co. Ltd. (Japan). Vitamin B3 compounds useful herein include, for example, those having the formula:

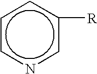

wherein R is —CONH₂ (e.g., niacinamide) or —CH₂OH (e.g., nicotinyl alcohol); derivatives thereof; and salts thereof. Exemplary derivatives of the foregoing vitamin B₃ compounds include nicotinic acid esters, including non-vasodilating esters of nicotinic acid, nicotinyl amino acids, nicotinyl alcohol esters of carboxylic acids, nicotinic acid N-oxide and niacinamide N-oxide. Preferred vitamin B₃ compounds are niacinamide and tocopherol nicotinate, and more preferred is niacinamide. In a preferred embodiment, the vitamin B₃ compound contains a limited amount of the salt form and is more preferably substantially free of salts of a vitamin B₃ compound. Preferably the vitamin B₃ compound contains less than about 50% of such salt, and is more preferably essentially free of the salt form. Commercially available vitamin B₃ compounds that are highly useful herein include niacinamide USP available from Reilly.

Vitamin B6 compounds useful herein include pyridoxine; esters of pyridoxine such as pyridoxine tripahnitate, pyridoxine dipalmitate, and pyridoxine dioctanoate; amines of pyridoxine such as pyridoxamine; salts of pyridoxine such as pyridoxine HCl; and derivatives thereof such as pyridoxamine, pyridoxal, pyridoxal phosphate, and pyridoxic acid. Particularly useful vitamin B6 compounds are selected from the group consisting of pyridoxine, esters of pyridoxine and salts of pyridoxine. The vitamin B6 compound can be synthetic or natural in origin and can be used as an essentially pure compound or mixtures of compounds (e.g., extracts from natural sources or mixtures of synthetic materials). As used herein, “vitamin B6” includes isomers and 6 tautomers of such. Commercially available vitamin B6 compound useful herein include, for example, pyridoxine HCl available from DSM, pyridoxine dipalmitate with tradename NIKKOL DP and pyridoxine dioctanoate with tradename NIKKOL DK available from Nikko Chemicals Co. Ltd.

Skin lightening agents useful herein refer to active ingredients that improve hyperpigmentation as compared to pre-treatment. Useful skin lightening agents herein include ascorbic acid compounds, acetyl glucosamine, azelaic acid, butyl hydroxyanisole, gallic acid and its derivatives, glycyrrhizinic acid, hydroquinone, kojic acid, arbutin, mulberry extract, and mixtures thereof. Use of combination of skin lightening agents is believed to be advantageous in that they may provide skin lightening benefit through different mechanisms.

Ascorbic acid compounds useful herein include, ascorbic acid per se in the L-form, ascorbic acid salt, and derivatives thereof. Ascorbic acid is available from, for example, Roche Vitamins Japan. Ascorbic acid salts useful herein include, sodium, potassium, lithium, calcium, magnesium, barium, ammonium and protamine salts. Ascorbic acid derivatives useful herein include, for example, esters of ascorbic acid, and ester salts of ascorbic acid. Particularly preferred ascorbic acid compounds include 2-o-D-glucopyranosyl-L-ascorbic acid, which is an ester of ascorbic acid and glucose and usually referred to as L-ascorbic acid 2-glucoside or ascorbyl glucoside, and its metal salts, and L-ascorbic acid phosphate ester salts such as sodium ascorbyl phosphate, potassium ascorbyl phosphate, magnesium ascorbyl phosphate, and calcium ascorbyl phosphate. Commercially available ascorbic compounds include magnesium ascorbyl phosphate available from Showa Denko, 2-o-D-glucopyranosyl-L-ascorbic acid available from Hayashibara and sodium L-ascorbyl phosphate with tradename STAY C50 available from DSM.

Other hydrophobic skin lightening agents useful herein include ascorbic acid derivatives such as ascorbyl tetraisopalmitate (for example, VC-IP available from Nikko Chemical), ascorbyl palmitate (for example available from DSM), ascorbyl dipalmitate (for example, NIKKOL CP available from Nikko Chemical); undecylenoyl phenyl alanine (for example, SEPIWHITE MSH available from Seppic); octadecenedioic acid (for example, ARLATONE DIOIC DCA available from Uniquema); ooenothera biennis sead extract, and pyrus malus (apple) fruit extract, and mixtures thereof.

Other skin benefit agents useful herein include those selected from the group consisting of panthenol, benzoyl peroxide, 3-hydroxy benzoic acid, farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (e.g., retinyl propionate), phytic acid, N-acetyl-L-cysteine, lipoic acid, tocopherol and its esters (e.g., tocopheryl acetate), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof.

UV protection agents for providing sunlight and UV protection benefit are useful as skin benefit agents herein. When included, the total of organic UV protection agent is from about 0.1% to about 20% of the capsule. Oil-soluble organic UV agents, water-soluble organic UV agents, and inorganic UV agents may be incorporated in the present capsule. Useful organic UV protection agents include both those which absorb UV radiation mainly in the UVB range, and those which absorb UV radiation mainly in the UVA range. Protection from UVB is described by SPF (Sun Protection Factor) and UVA is described by PA (Protection of UVA). It is well known in the art that combining UVA and UVB protection agents provide a composition having effective sunscreen effect. In one preferred embodiment, the present invention is a sunscreen product or a cosmetic product having an SPF of at least 15 and a PA of at least ++.

Useful oil-soluble organic UV protection agents effective as UVB filters include: 3-benzylidenecamphor derivatives, preferably 3-(4-methylbenzylidene) camphor and 3-benzylidenecamphor; aminobenzoic acid derivatives, preferably 2-ethylhexyl 4-(dimethylamino)-benzoate and amyl 4-(dimethyl amino) benzoate; esters of cinnamic acid, preferably 2-ethylhexyl 4-methoxycinnamate and isopentyl 4-methoxycinnamate; esters of salicylic acid, preferably 2-thylhexyl salicylate, 4-isopropylbenzyl salicylate and homomethyl salicylate; derivatives of benzophenone, preferably 2-hydroxy-4-methoxybenzophenone (Benzophenone-3), 2-hydroxy-4-methoxy-4′-methyl benzophenone and 2.2′-dihydroxy-4-methoxybenzophenone; esters of benzalmalonic acid, preferably di(2-ethylhexyl) 4-methoxybenzalmalonate; and 2,4,6-trianilino-(p-carbo-2′-ethyl-1′-hexyloxy)-1,3,5-triazine.

Useful oil-soluble organic UV protection agents effective as UVA filters include: derivatives of dibenzoylmethane, in particular 1-(4′-tert-butylphenyl)-3-(4′-methoxyphenyl) propane-1.3-dione and 1-phenyl-3-(4′-isopropylphenyl) propane-1.3-dione.

Commercially available oil-soluble organic UV protection agents herein include: 2-ethylhexyl 4-methoxycinnamate with tradename PARSOL MCX available from ROCHE VITAMINS JAPAN K.K and 2-hydroxy-4-methoxybenzophenone (Benzophenone-3) available from BASF.

Useful water-soluble organic UV protection agents effective as UVB filters include: 2-phenylbenzimidazole-5-sulphonic acid, and its sodium, potassium or its triethanol-ammonium salts; sulphonic acid derivatives of benzophenones, preferably 2-hydroxy-4-methoxybenzophenone-5-sulphonic acid (Benzophenone-4) and its salts; sulphonic acid derivatives of 3-benzylidenecamphor, such as, for example 4-(2-oxo-3-bornylidenemethyl)-benzenesulphonic acid, 2-methyl-5-(2-oxo-3-bornylidenemethyl) sulphonic acid and its salts.

Commercially available water-soluble organic UV protection agents herein include: phenylbenzimidazole-5-sulphonic acid with tradename PARSOL HS available from BASF and Neo Helopan Hydro available from Symrise, and 2-hydroxy-4-methoxybenzophenone-5-sulphonic acid (Benzophenone-4) available from BASF.

Useful inorganic UV protection agents herein are cosmetic and dermatological acceptable metal oxides and/or other metal compounds which are sparingly soluble or insoluble in water, in particular the oxides of titanium (TiO₂), zinc (ZnO), iron (for example Fe₂O₃), zirconium (ZrO₂), silicon (SiO₂), manganese (for example MnO), aluminum (Al₂O₃) and cerium (for example Ce₂O₃), mixed oxides of the corresponding metals and mixtures of such oxides. Inorganic UV protection agents have a particle size of smaller than 200 nm, preferably smaller than 100 nm. Thus, depending on the surface coating characteristic, the spindle-shaped metal oxide powders and certain color powders described above may provide UV protection benefit. Those that are not surface coated with hydrophobic coating material are also useful herein as UV protection agents that disperse in the water phase.

Commercially available inorganic UV protection agents herein include: zinc oxide having an average particle size of about 70 nm with tradename Z-cote HP1 available from BASF, and titanium oxide having an average particle size of about 50 nm with tradenames SI-TTO-S-3Z-LHC and SAMT-UFZO-450 available from Miyoshi, and Zinc Oxide coated with Triethoxycaprylylsilane having a particle size of about 20 nm with tradename OTS-7 FZO-50 available from Daito Kasei.

### Additional Components

The capsules herein may further contain additional components conventionally used in topical products, e.g., for providing aesthetic or functional benefit to the composition or personal surface, such as sensory benefits relating to appearance, smell, or feel, therapeutic benefits, or prophylactic benefits (it is to be understood that the above-described required materials may themselves provide such benefits). When included, the amount is kept to no more than about 10% by weight of the capsule.

Examples of suitable topical ingredient classes include: powders and pigments that do not meet the definition of other components described above, anti-chelating agents, abrasives, astringents, dyes, essential oils, fragrance, film forming polymers, solubilizing agents, anti-caking agents, antifoaming agents, binders, buffering agents, bulking agents, denaturants, pH adjusters, propellants, reducing agents, sequestrants, cosmetic biocides, and preservatives.

### Process for Making the Collapsible Water-Containing Capsule

The present invention relates to suitable processes for making the collapsible water-containing capsules as described above in an economical and effective manner, while the physical structures of the capsules are maintained. The process relates to mixing the water phase and the powder phase, the powder phase comprising the spindle-shaped metal oxide powder, optional spherical powder, and optional color powder. For convenience, in this section, the mixing of the water phase and the powder phase for forming the capsule is referred to as “main mixing”, while mixing of certain compositional components prior to the main mixing is referred to as “premixing”.

As described above, without being bound by theory, it is believed that, by the surface tension of the surface of the spindle-shaped metal oxide powder, the spindle-shaped metal oxide powder aligns at the phase boundary of the water phase, while the particles of the spindle-shaped metal oxide powder bind with each other via van-der-Waals binding. The remaining larger optional spherical powders and color powders align at the outer boundary of the spindle-shaped metal oxide powder. The suitable processes herein are those which provide enough energy to micronize the water phase and to maintain the size of the micronized water phase, and thus allow the spindle-shaped metal oxide powders to align at the phase boundary to form a stable capsule, yet do not provide the shear stress that would immediately destroy the physical structure of the capsule. Preferably avoided are means that apply high shear stress to the capsules, such as high speed agitation, and mechanical mixing means which provide crushing or kneading.

Generally, the water phase and the powder phase are separately prepared prior to main mixing. The powder phase may be pulverized to fragment any agglomeration which may interfere with the following capsule making process. When gelling agents are incorporated, the gelling agent may be premixed with either the remainder of the water phase or the powder phase, depending on the physical properties of the compositional components, and the components of the mixing apparatus.

In one preferred embodiment, the inner wall of the vessel for main mixing is hydrophobically coated with, for example, silicone or Teflon, to lower the surface energy of the inner wall, and thereby provide the capsule making in an efficient manner. When a final primary package is directly used for main mixing, as detailed below as the “make-in-pack” process, the inner wall of the final primary package should have a surface energy of 50 dyne/cm or less, preferably 40 dyne/cm or less.

Suitable mixing apparatus for the main mixing are the external energy sourcing type or container shaking type. These apparatus are those which do not have a mixing blade or the like within the vessel in which the capsule is made. These apparatus are advantageous in that there is hardly any, or only a controllable amount of shear stress provided during the making process. These apparatus are also advantageous in that the making process is done in a relatively short length of time.

Mixing apparatus of the external energy sourcing type include, but are not limited to, vibratory mixer, and resonant frequency mixer. Vibratory mixers are those that provide convection mixing by impact of vertical shaking motion, gyroscopic oscillating or vibration frequency. Resonant frequency mixers are those that use an oscillator to excite the material for mixing by high efficient energy transfer. Mixing apparatus of the container shaking type are those that do not provide rotating movement, but provide convection mixing by impact of alternative acceleration or retardation of gyroscopic shaking motion. In these external energy sourcing type or container shaking type apparatus, the compositional components for making the capsule are simply filled in the mixing vessel together, and mixed. The mixing vessel is not inverted. Thus, these apparatus may be used for providing a process wherein the capsule is directly made in a final primary packaging for consumer use, the so-called “make-in-pack” process. Accordingly, in one highly preferred embodiment, the present process relates to the use of a mixing apparatus of the external energy sourcing type or container shaking type, wherein the capsule is to be provided in a final primary packaging for consumer use, wherein the process comprises the steps of:
i) directly supplying the water phase and the spindle-shaped metal oxide powder in the final primary packaging; and
ii) mounting the product of step i) onto the mixing apparatus for making the capsule.

Herein, what is meant by the final primary packaging is the primary packaging in which the consumer receives the product, rather than an interim vessel or package which is only used for delivering or filling the product into a final primary package.

Commercially available vibratory mixers highly preferred herein include COROB 200 available from CPS Color, and TSTM Vibratory Mixer and Vibratory Mixer Type 1 available from Tsukishima Techno Machinery Co., Ltd. Commercially available resonant frequency mixers highly preferred herein include Resodyn Resonant Acoustic Mixers available from Resodyn Corporation. Commercially available container shaker mixers highly preferred herein include TURBULA Mixer Type T2F, T10B, T50A, and Dyna Mix available from Willy A. Bachofen AG, and COROB M300/CORB and VIBRO available from CPS Color.

In another embodiment, the present capsule is provided to the consumer as a preparation-at-use product for providing a collapsible water-containing capsule comprising the compositional components of the capsule and a final primary packaging having an inner wall having a surface tension of 50 dyne/cm or less;

wherein the water phase and the spindle-shaped metal oxide powder are separately packaged prior to use, and wherein the capsule is made by the steps of;
i) filling the water phase and the spindle-shaped metal oxide powder into the final primary packaging; and
ii) manually shaking the product of step i) until the water phase is encapsulated in the spindle-shaped metal oxide powder.

In this embodiment, the capsule making process happens at use by manual shaking of the final primary packaging by the user. Such preparation-at-use product provides the user of the feeling that the product is freshly made upon use, and/or the amusement of making the product. Alternatively, such preparation-at-use action may be used as an effective demonstration of making the product for market promotion or otherwise.

Regardless of the mixing apparatus used for providing the capsule, the completion of encapsulation can be determined by identifying powder like appearance by the naked eye, with no liquid remaining in the container in which the mixing was conducted.

### Shock Stability of the Collapsible Water-Containing Capsule

The present capsule has appropriate shock stability such that it is stable under normal storage conditions as well as normal mixing processes, however, collapses upon application on the personal surface. What is meant by normal storage condition, is an environment of 5° C. to 40° C. The collapsing of the present capsule can be easily observed by the naked eye, as a flowable dry powdery appearance of the original capsule is changed to a non-flowable wet pasty appearance.

Such shock stability is suitably quantitatively measured by the Tumbling Impact Method as described in the Example section below. The present capsule preferably has a shock stability of at least 8 minutes. The shock stability may be adjusted according to the proposed usage of the product. It is possible, according to the present invention to provide capsules that have very high shock stability, however, such stability must be balanced with the collapsibility upon application, and preferred cooling sensation upon collapse.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

The following are capsule compositions for use on skin, method of preparation thereof, and technical and sensory assessment of their characteristics thereof. Examples 1-5 are capsules according to the present invention, while Comparative Examples 1-4 are those that are not according to the present invention. Further, Reference Example 1 is provided for characterizing the preferred water repelling silicone elastomer powder herein.

### Reference Example 1

The water repelling silicone elastomer powder utilized in Examples below are prepared as such.

In a 1 liter glass beaker, 500 g of methylvinylpolysiloxane of formula (1) having a viscosity of 580 mm2/s and 19 g of methylhydrogenpolysiloxane of formula (2) having a viscosity of 30 mm2/s (namely, an amount wherein the number of hydrosilyl group is 1.06 per every olefin unsaturated group) were dissolved by mixing via a homomixer at 2000 rpm. Then, 3 g of polyoxyethylenelaurylether (9 mols of added ethyleneoxide) and 55 g of water was added and mixed with a homomixer at 6000 rpm to achieve an oil-in-water emulsion form and viscosifying, and further mixed for 15 minutes. Then, by adding 421 g of water under mixing at 2000 rpm, a homogenous white emulsion was obtained. This emulsion was transferred to a 1 liter glass flask having a mixing apparatus with an anchor mixing blade, adjusted to a temperature of 15-20° C., added with a co-solution of 0.8 g of toluene solution of chloroplatinic acid olefin complex (having platinum content of 0.5%) and 1.6 g of polyoxyethylenelaurylether (9 mols of added ethyleneoxide), and mixed at the same temperature for 12 hrs, to obtain a water dispersion of fine particles of silicone elastomer. The silicone elastomer fine particles were spherical in shape by observing by optical microscope, and had a volume average particle size of 5 μm by measuring with an electric resistance method particle distribution measuring device “Multisizer-3” (Beckman Coulter).

870 g of such obtained water dispersant of spherical silicone elastomer fine particles were transferred to a 3 liter glass flask having a mixing apparatus with an anchor mixing blade, and added with 2013 g of water and 57 g of 28% ammonia solution. The pH of this fluid was 11.3. After adjusting the temperature to 5-10° C., 46.8 g of methyltrimethoxysilane (for 100 weight parts of spherical elastomer fine particle, 5.1 weight parts of hydrolytically condensed polymethylsilsesquioxane) was dropped over a period of 20 minutes while keeping the fluid temperature at 5-10° C., then 8.4 g of trimethylsilanol (for 100 weight parts of spherical elastomer fine particle, 1.9 weight parts of hydrolytically condensed polymethylsilsesquioxane) and 4.8 g of tetramethoxysilane (0.34 mols per 1 mol of trimethylsilanol) was dropped over a period of 5 minutes while keeping the fluid temperature at 5-10° C., mixed at the same temperature for another 1 hr, to complete the hydrolytic condensation of methyltrimethoxysilane, tetramethoxysilane, and trimethylsilanol.

The hydrolytic condensate fluid of methyltrimethoxysilane, tetramethoxysilane, and trimethylsilanol methoxysilyl in the water dispersion of silicone elastomer fine particle was dehydrated with a pressurized filter to water content of about 30%. The dehydrate was transferred to a 5 liter glass flask having a mixing apparatus with an anchor mixing blade, added with 3000 g of 50% methanol solution and mixed for 30 minutes, and dehydrated with a pressurized filter. The dehydrate was transferred to a 5 liter glass flask having a mixing apparatus with an anchor mixing blade, added with 3000 g of water and mixed for 30 minutes, and dehydrated with a pressurized filter. The dehydrate was dried at 105° C. in a hot air convention drier and crushed in a jet mill, to obtain a fluid fine particle. By observing with an electronic microscope, it was confirmed that the obtained was a spherical fine particle surface coated with particulates of about 100 nm, wherein the spherical silicone elastomer fine particle was coated with polymethylsilsequioxane. By dispersing the fine particles in water using surfactant and measured by measuring with an electric resistance method particle distribution measuring device “Multisizer-3” (Beckman Coulter), the volume average particle size was 5 μm. When measured by JIS K 6253, the obtained fine particles had a Durometer A Hardness of 29.

When 1 g of the obtained fine particles were placed in an 100 ml beaker with 50 g of water and mixed for 1 minutes with a glass rod, none of the particles dispersed in water, but remained floating at the surface.

TABLE 1
Compositions and Test Results for Examples 1-5

| | Components | Ex. 1 | Ex. 2 | Ex. 3 | Ex. 4 | Ex. 5 |
|---|---|---|---|---|---|---|
| A | Spindle-shaped Titanium Dioxide coated with | 13 | 9.5 | 13 | 18 | 9 |
| | Triethoxycaprylylsilane (10 nm/60 nm, aspect ratio 6) | | | | | |
| | *1 | | | | | |
| A | Trimethylsilyl Vinyl Dimethicone/Methicone | 10 | 10 | | 7 | 5.6 |
| | Silsesquioxane Crosspolymer of Reference Example 1 | | | | | |
| A | Methyl Methacrylate Crosspolymer coated with | | | | 1.62 | |
| | Triethoxycaprylylsilane (5 μm) *2 | | | | | |
| A | DL-alpha-Tocopheryl Acetate containing Silica coated | 0.2 | | 0.2 | | |
| | with Dimethicone (5 μm) *3 | | | | | |
| A | Titanium Dioxide coated with Triethoxycaprylylsilane | 1 | 5 | 1 | | |
| | (250 nm) *4 | | | | | |
| A | Zinc Oxide coated with Triethoxycaprylylsilane | | 2 | | | |
| | (20 nm, amorphous) *5 | | | | | |
| A | Yellow Iron Oxide coated with | 0.35 | 1.5 | 0.35 | | |
| | Triethoxycaprylylsilane (400 nm) *6 | | | | | |
| A | Black Iron Oxide coated with Triethoxycaprylylsilane | 0.1 | 0.6 | 0.1 | | |
| | (400 nm) *7 | | | | | |
| A | Red Iron Oxide coated with Triethoxycaprylylsilane | 0.1 | 0.6 | 0.1 | | |
| | (400 nm) *8 | | | | | |
| A | Mica coated with Triethoxycaprylylsilan (20 μm) *9 | 1.87 | 5 | 11.87 | | |
| A | Mica, Titanium Dioxide coated with Dimethicone | | | | | 2.5 |
| | (20 μm) *10 | | | | | |
| A | Aluminum Oxide, Titanium Dioxide and Tin coated | | | | | 2.5 |
| | with Dimethicone (20 μm) *11 | | | | | |
| A | Ascorbic Acid *12 | | 1 | | | |
| A | Fragrance | | 0.01 | | | |
| B | Sodium Carboxymethyl Starch *13 | 0.5 | 0.5 | 0.5 | | |
| B | Sodium Polyacrylate *14 | | | | 0.1 | 0.1 |
| B | Ethanol | | | | 1 | |
| B | Titanium Dioxide coated with Microcrystalline | | | | | 1 |
| | Cellulose and Cellulose Gum (250 nm) *15 | | | | | |
| B | Mica coated with Titanium Dioxide (20 μm) *16 | | | | | 1 |
| B | Galactomyces Ferment Filtrate *17 | | 5 | | 10 | |
| B | Butylene Glycol *18 | | | | | 10 |
| B | Glycerin | 15 | | 15 | 5 | |
| B | Niacinamide *19 | 3.5 | | 3.5 | 5 | |
| B | Mulberry Root Extract *20 | | | | 3 | |
| B | Acetyl Glucosamine *21 | | | | 1 | |
| B | EDTA-2NA | | | | 0.1 | |
| B | DL-Panthenol *22 | 1.12 | | 1.12 | | |
| B | Preservative | 0.7 | 0.7 | 0.7 | 0.7 | 0.7 |
| B | DE-IONIZED WATER | 52.56 | 58.59 | 52.56 | 47.48 | 67.6 |
| | Total | 100 | 100 | 100 | 100 | 100 |
| | Capsulation | Good | Good | Good | Good | Good |
| | Shock Stability | 12 | 15 | 8 | 9 | 10 |
| | Cooling Sensory on Application | 4.4 | 3.4 | 4.4 | 3.6 | 3.8 |

TABLE 2
Compositions and Test Results for Comparative Examples 1-4

| | | Com. | Com. | Com. | Com. |
|---|---|---|---|---|---|
| | Components | Ex. 1 | Ex. 2 | Ex. 3 | Ex. 4 |
| A | Spindle-shaped Titanium Dioxide coated with | | | 3 | 13 |
| | Triethoxycaprylylsilane (10 nm/60 nm, aspect ratio 6) *1 | | | | |
| A | Titanium Dioxide coated with Triethoxycaprylylsilane | 13 | | | |
| | (35 nm, amorphous) *23 | | | | |
| A | Silica Dimethyl Silylate (15 nm, porous) *24 | | 13 | | |
| A | Trimethylsilyl Vinyl Dimethicone/Methicone | 10 | 10 | 10 | 10 |
| | Silsesquioxane Crosspolymer of Reference Example 1 | | | | |
| A | DL-alpha-Tocopheryl Acetate containing Silica coated | 0.2 | 0.2 | 0.2 | 0.2 |
| | with Dimethicone (5 μm) *3 | | | | |
| A | Titanium Dioxide coated with Triethoxycaprylylsilane | 1 | 1 | 1 | 1 |
| | (250 nm) *4 | | | | |
| A | Yellow Iron Oxide coated with | 0.35 | 0.35 | 0.35 | 0.35 |
| | Triethoxycaprylylsilane (400 nm) *6 | | | | |
| A | Black Iron Oxide coated with Triethoxycaprylylsilane | 0.1 | 0.1 | 0.1 | 0.1 |
| | (400 nm) *7 | | | | |
| A | Red Iron Oxide coated with Triethoxycaprylylsilane | 0.1 | 0.1 | 0.1 | 0.1 |
| | (400 nm) *8 | | | | |
| A | Mica coated with Triethoxycaprylylsilan (20 μm) *9 | 1.87 | 1.87 | 11.87 | 41.87 |
| B | Sodium Carboxymethyl Starch *13 | 0.5 | 0.5 | 0.5 | 0.5 |
| B | Glycerin | 15 | 15 | 15 | 15 |
| B | Niacinamide *19 | 3.5 | 3.5 | 3.5 | 3.5 |
| B | DL-Panthenol *22 | 1.12 | 1.12 | 1.12 | 1.12 |
| B | Preservative | 0.7 | 0.7 | 0.7 | 0.7 |
| B | DE-IONIZED WATER | 52.56 | 52.56 | 52.56 | 12.56 |
| | Total | 100 | 100 | 100 | 100 |
| | Capsulation | Not | Good | Good | Good |
| | | Good | | | |
| | Shock Stability | N/A | 15 | 2 | 15 |
| | Cooling Sensory on Application | N/A | 0.2 | 4.4 | 0.8 |

Definitions of Components
*1 Spindle-shaped Titanium Dioxide coated with Triethoxycaprylylsilane (10 nm/60 nm, aspect ratio 6): OTS-11 TTO-V-3 available from Daito Kasei.
*2 Methyl Methacrylate Crosspolymer coated with Triethoxycaprylylsilane (5 μm): OTS-2 MR-7GC available from Daito Kasei.
*3 DL-alpha-Tocopheryl Acetate containing Silica coated with Dimethicone (5 μm): SA-SB-705/VEAC(50%) available from Miyoshi Kasei.
*4 Titanium Dioxide coated with Triethoxycaprylylsilane (250 nm): OTS-2 TiO2 CR-50 available from Daito Kasei.
*5 Zinc Oxide coated with Triethoxycaprylylsilane (20 nm, amorphous): OTS-7 FZO-50 available from Daito Kasei
*6 Yellow Iron Oxide coated with Triethoxycaprylylsilane (400 nm): OTS-2 YELLOW LL-100P available from Daito Kasei.
*7 Black Iron Oxide coated with Triethoxycaprylylsilane (400 nm): OTS-2 BLACK BL-100P available from Daito Kasei.
*8 Red Iron Oxide coated with Triethoxycaprylylsilane (400 nm): OTS-2 RED R-516P available from Daito Kasei.
*9 Mica coated with Triethoxycaprylylsilan (20 μm): OTS-2 MICA Y-2300 available from Daito Kasei.
*10 Mica, Titanium Dioxide coated with Dimethicone (20 μm): SA FLAMENCO RED available from Miyoshi Kasei.
*11 Aluminum Oxide, Titanium Dioxide and Tin coated with Dimethicone (20 μm): SA Xirona Silver available from Miyoshi Kasei.
*12 Ascorbic Acid: Ascorbic Acid available from ROCHE VITAMINS JAPAN K.K.
*13 Sodium Carboxymethyl Starch: COVAGEL available from LCW.
*14 Sodium Polyacrylate: COVACRYL MV60 available from LCW.
*15 Titanium Dioxide coated with Microcrystalline Cellulose and Cellulose Gum (250 nm): AC-5 TiO2 CR-50 available from Daito Kasei.
*16 Mica coated with Titanium Dioxide (20 μm): FLAMENCO SUPER PEARL available from THE MEARL.
*17 Galactomyces Ferment Filtrate: SK-II Pitera available from Kashiwayama.
*18 Butylene Glycol: 1,3-Butylene Glycol available from Celanese.
*19 Niacinamide: Niacinamide USP available from DSM.
*20 Mulberry Root Extract: Mulberry BG, available from Maruzen Pharmaceuticals.
*21 Acetyl Glucosamine: N-Acetyl-D-glucosamine, available from Technical Sourcing International.
*22 DL-Panthenol: D-Panthenol USP, available from DSM
*23 Titanium Dioxide coated with Triethoxycaprylylsilane (35 nm, amorphous): OTS-5 TiO2 MT500SA available from Daito Kasei.
*24 Silica Dimethyl Silylate (15 nm, porous): Aerosil R 972 available from Nihon Aerosil.

### Method of Preparation

Components A are mixed and transferred to a container that has a hydrophobic inner surface. Components B are separately mixed and transferred to the same container. The container is closed and shook by TURBLER Mixer Type T2F (Willy A. Bachofen AG) at 95 rpm for 3 min.

### Methods of Tests

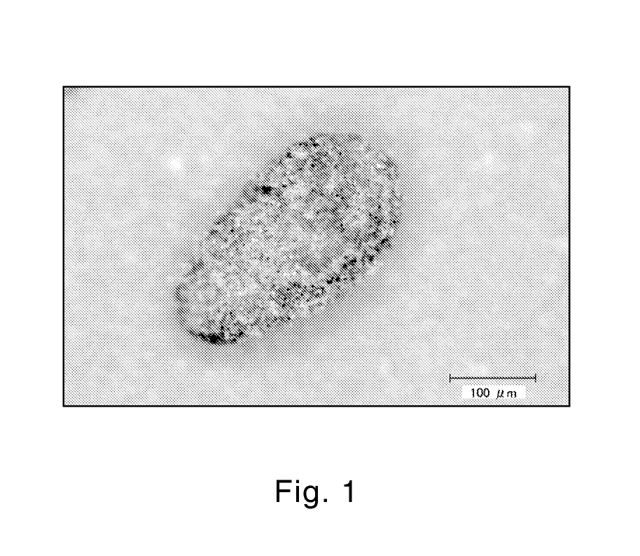

Capsulation: If capsules of even fine particles are observed by DIGITAL MICROSCOPE VHX-900 from KEYENCE, evaluation is “Good”. If the capsules are not formed, evaluation is “Not Good”. FIG. 1 provides a microscopic photograph at a magnitude of 200 times of a capsule of a preferred embodiment of the present invention that has been successfully formed. As can be seen from FIG. 1, a clear boundary of the capsule is observed. When the capsule is not formed, such boundary is not observed, but rather a more or less homogenous mass is observed. For those compositions that did not form capsules, it is not possible to conduct the remaining tests.

Shock Stability (Tumbling Impact Method): 5 g of powder sample is weighed and placed in a 50 ml polypropylene container. After closing a cap, put the container into 1 L plastic container. The 1 L container is capped and set on a TURBLER Mixer Type T2F (Willy A. Bachofen AG), and shook at 100 rpm for 1 min, and stopped for observation. The same shaking and observation procedure is repeated after each minute of shaking until a total of 15 cycles. If the powder sample is collapsed and changed to liquid, it is considered end point and total shaking time is recorded. If the sample endures shaking for total 5 minutes and collapsed at total 6 minutes, the value is defined as “5 minutes”. Those compositions enduring 8 minutes of shaking are considered as having acceptable stability.

Cooling Sensory on Application: Cooling Sensory is evaluated upon application on the hand by five expert panelists with 5 scale grades (No Cooling-1, Very Weak Cooling-2, Weak Cooling-3, Strong Cooling-4 and Very Strong Cooling-5). Then average is calculated. Those compositions that do not provide more than a calculated score of 3.0 are considered as not providing satisfactory cooling sensation.

### Evaluation

The test results of Examples 1-5 and Comparative Examples 1-4 are found in Tables 1 and 2. Comparative Example 1 devoid of the spindle-shaped metal oxide powder, and containing an amorphous shaped titanium dioxide instead, did not form a capsule. Comparative Example 2 devoid of the spindle-shaped metal oxide powder, and containing porous submicron (15 nm) sized silica dimethyl silylate instead, did not provide acceptable sensory benefits. Comparative Example 3 having less than the required amount of the spindle-shaped metal oxide powder, but compensated with amorphous shaped titanium dioxide instead, did not provide acceptable stability. Comparative Example 4 having less than the required amount of water phase did not provide acceptable sensory benefits. Example 3 devoid of spherical powder provided acceptable stability, however, other Examples containing spherical powder provided better stability.

### Usage of Examples 1-5

The capsules of Examples 1-5 have appropriate shock stability such that it is stable under normal storage conditions as well as normal mixing processes, however, collapses upon a certain shear stress upon application on the skin. When collapsed, the capsules of Examples 1-5 provide good feel to the skin. Examples 1-3 are useful as foundations. When applied on the skin, the capsules provide suitable cooling sensation, good appearance on the skin by balanced coverage and natural look. Example 4 is useful as a skin lightening powder and/or cooling powder. When applied on the skin, the capsules provide suitable cooling sensation, and the skin lightening agents are penetrated to the skin. Example 5 is useful as an eye shadow and blusher. When applied on the skin, the capsules provide suitable cooling sensation and good look.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A collapsible water-containing capsule comprising by weight:
(a) from about 40% to about 95% of a water phase comprising at least 50% water by weight of the water phase; and
(b) from about 5% to about 20% of a spindle-shaped metal oxide powder which is hydrophobically surface-treated and has an average long axis particle size of from about 25 nm to about 150 nm, an average short axis particle size of from about 4 nm to about 50 nm, and an aspect ratio of greater than about 3.

2. The capsule of claim 1 wherein the spindle-shaped metal oxide is titanium dioxide.

3. The capsule of claim 1 further comprising a spherical powder having an average particle size of at least 1 μm and which has a hydrophobic surface.

4. The capsule of claim 3 wherein the spherical powder is a silicone elastomer powder.

5. The capsule of claim 4 wherein the silicone elastomer powder is a water repelling silicone elastomer powder comprising 100 weight parts of a spherical silicone elastomer particle and 0.5-25 weight parts of polyorganosilsequioxane for coating the spherical silicone elastomer particle; wherein the water repelling silicone elastomer powder does not disperse in, but floats in water; has an average particle size of at least 1 μm and has a softness of from about 10 to about 80 measured by Durometer A Hardness.

6. The capsule of claim 1 further comprising from about 0.1% to about 8% of a color powder having an average particle size of from about 0.15 μm to less than 1 μm and which is hydrophobically surface-treated.

7. The capsule of claim 1 wherein the water phase further comprises a gelling agent.

8. The capsule of claim 1 further comprising a skin benefit agent.

9. The capsule of claim 1 further comprising a perfume.

10. The capsule of claim 1 wherein the capsule is substantially free of surfactants.

11. The capsule of claim 1 wherein the capsule comprises less than 1% of porous powders having a particle size of less than 1 μm.

12. The capsule of claim 1 wherein the capsule is substantially free of fluorine surface coated pigments.

13. A cosmetic composition comprising the capsule of claim 1.

14. The capsule of claim 1 wherein the capsule has appropriate shock stability such that it is stable under normal storage conditions as well as normal mixing processes, however, collapses upon application on the personal surface.

15. The capsule of claim 14 wherein the capsule has a shock stability of at least 8 minutes when measured by the Tumbling Impact Method herein defined.

16. A process for making the capsule of claim 1 wherein the components of the capsule are mixed by a mixing apparatus selected from the group consisting of external energy sourcing type and container shaking type.

17. The process of claim 16 wherein the capsule is to be provided in a final primary packaging for consumer use, wherein the process comprises the steps of:
i) directly supplying the water phase and the spindle-shaped metal oxide powder in the final primary packaging; and
ii) mounting the product of step i) onto the mixing apparatus for making the capsule.

18. A preparation-at-use product for providing the collapsible water-containing capsule of claim 1 comprising the compositional components of the capsule and a final primary packaging having an inner wall having a surface tension of 50 dyne/cm or less;
wherein the water phase and the spindle-shaped metal oxide powder are separately packaged prior to use, and wherein the capsule is made by the steps of:
i) filling the water phase and the spindle-shaped metal oxide powder into the final primary packaging; and
ii) manually shaking the product of step i) until the water phase is encapsulated in the spindle-shaped metal oxide powder.

19. A method of treating or making up of the skin comprising the steps of:
(1) viding the collapsible water-containing capsule of claim 1;
(2) shearing the collapsible water-containing capsule on the skin by a finger or an applicator to allow the collapsible water-containing capsule to collapse; whereby the components of the collapsible water-containing capsule are applied on the skin; and
(3) allowing the water to evaporate and/or be absorbed in the skin.
